(19) United States
(12) Patent Application Publication
(10) Pub. No.: US 2003/0167048 A1
(43) Pub. Date: Sep. 4, 2003

(54) Comfortable tampon

(76) Inventor: Nini Policappelli, Los Angeles, CA (US)

Correspondence Address: GREENBERG TRAURIG LLP, 2450 COLORADO AVENUE, SUITE 400E, SANTA MONICA, CA, 90404, US

(21) Appl. No.: 10/373,541

(22) Filed: Feb. 24, 2003

Related U.S. Application Data
(60) Provisional application No. 60/361,164, filed on Mar. 1, 2002.

(51) Int. Cl. A61F 13/20

(52) U.S. Cl. 604/385.17; 604/904

(57) ABSTRACT

A tampon comprising an elongated absorptive area and an elongated element within the absorptive area which is expandable on insertion of air. This increases the comfort at the interface of the tampon with the vagina walls. Optionally, a plurality of nonabsorptive elements are located on the outside surface of the tampon material, the nonabsorptive elements causing a spacing between the absorptive elements and vagina wall.

### BACKGROUND OF THE INVENTION

### FIELD OF THE INVENTION

The background of this invention relates to a tampon. In particular, the invention is concerned with a tampon with improved comfort for the user.

Many different forms and shapes of tampons are known. Because of the different anatomical features of users, there is often a problem in the comfort of the tampon when in use.

This invention is directed to providing a tampon with increased comfort but at the same time permitting effective absorption of bodily fluids in the absorptive portions of the tampon, and to minimize infection and toxic shock syndrome.

### SUMMARY OF THE INVENTION

According to the invention, there is provided a tampon which comprises an elongated absorptive element which has a central axis. This is for location in the vagina. The elongated element has at least one elongated area which is expandable on the insertion of a fluid into the expandable area. This causes the absorptive area generally to be edged outwardly from the central axis into a relatively closer engagement with the wall of the vagina.

In a preferred form of the invention, the expandable area is centrally located and includes a plug located at a mouth of the centrally located area. The mouth is at the end of the element for location adjacent the entrance to the vagina.

In yet a further preferred form of the invention, the plug includes a pump, the pumping for selectively expanding the elongated area by the injection of fluid into the elongated area.

The elongated area and the pump form a selectively closed chamber. The pump means includes a valve for permitting fluid to pass into the elongated chamber and selectively permitting fluid to exit from the closed chamber.

In the preferred form of the invention, the fluid is air and the pump acts to permit air to pass into the chamber in the first mode of operation. In the second mode of operation, the valve permits the air to exit the chamber.

The outside surface of the elongated absorptive area includes several nonabsorptive elements spaced about this surface. The nonabsorptive elements are spaced on the outside surface of the absorptive element and cause the absorptive element to be relatively spaced from the walls of the vagina. This effectively permits the walls of the vagina, which would otherwise normally be in contact with the absorptive element, to have a different interaction with the nonabsorptive elements and thereby provide for a different comfort field of the tampon in the vagina.

The nonabsorptive elements on the absorptive elements may be used with a tampon even though there is no pump associated with the tampon.

The non-absorbative elements can be a sleeve to locate over an existing tampon. The sleeve may be formed of a suitable silicon and may be suitably lubricated.

### BRIEF DESCRIPTION OF THE DRAWINGS

FIG. 1 is a side view of a tampon with the pump at the mouth of the elongated aperture.

FIG. 2 is a cross-sectional view showing the absorptive materials in the central aperture within the absorptive material in a plug located at the mouth of the central aperture. Cross-sectional diagonal elements made of silicon selectively lubricated, will fit in slots about the absorptive material.

FIG. 3 is a side view of a tampon with diagonally directed silicon elements of an absorptive material. There is no central aperture or plug in that configuration.

FIG. 4 is a side view showing a diagonal elastic silicon sleeve element formed like a sleeve and fitted over the absorptive elements and there is a central expandable portion fitted with a plug.

FIG. 5 is a side view of a different formation of elastic-like silicon elements over the absorptive elements and a central expandable area with a plug at the mouth of the central area.

FIG. 6 is a side view of a tampon without a plug and an elastic silicon network over the absorptive elements.

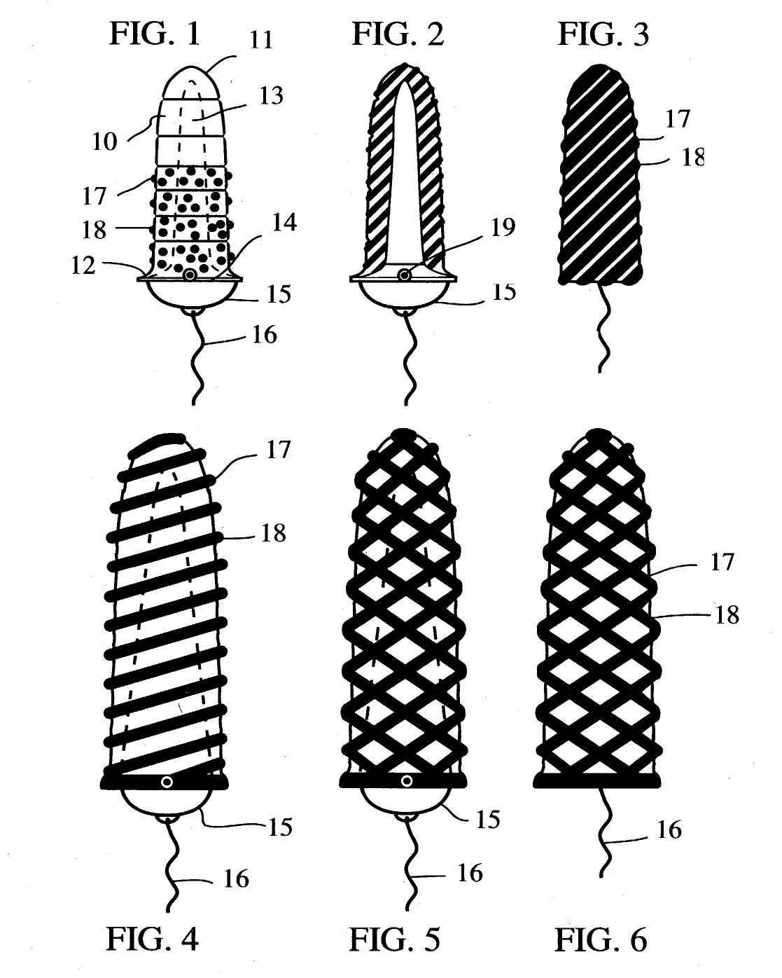

### DETAILED DESCRIPTION OF THE PREFERRED EMBODIMENTS

In the following description of the preferred embodiments reference is made to the accompanying drawings which form the part thereof, and in which are shown by way of illustration of specific embodiments in which the invention may be practiced. It is to be understood that other embodiments may be utilized and structural and functional changes may be made without departing from the scope of the present invention.

A tampon includes an elongated layer 10 of one or more layers of absorptive material such as cotton, other absorbative materials, or the like. The lead end 11 of the tampon is for insertion into the vagina and the trailing end 12 is located proximal the mouth of the vagina.

A central elongated portion 13 is located inside the absorptive material 10 and there is an open end or mouth 14 to the central area. A plug 15 closes the mouth and the free end of the plug is connected with a string or cord 16 to facilitate withdrawal of the tampon from the vagina. The plug 15 can include a pump configuration to permit air to be pumped from outside of the pump into the central axial area 13 and thereby cause that area 13 to expand. One or more valves 19 can be provided between the pump 15 and the central area 13 so that the air pumped into the central area 13 can be retained. Under appropriate circumstances, the valve 19 can operate in the reverse direction to permit air to be withdrawn from the central area.

The outside surface of the absorptive material 10 includes multiple lubricated silicon-like elements 17 and 18 arranged on the outside surface. These silicon materials 17 and 18 improve.the comfort when the tampon is located in the vagina. They are to protect the cotton which is located underneath and between the silicon elements, and also the internal parts of the vagina from the cotton. As shown in FIG. 2, the silicon elements 17 and 18 form a spiral about. the outside of the absorptive material. There is also a pump 15 with the open end of the central area of the tampon.

In FIG. 3 there is a silicon spiral member I7, 18 and there is no pump in that configuration of the tampon.

In FIG. 4 the spiral elements 17, 18 have a different angular relationship to the absorptive materials of the tampon. That configuration uses a pump 15.

A meshed arrangement is shown in FIG. 5 with a pump 15.

In FIG. 6 there is a similar meshed arrangement 17, 18 without a pump 15.

The silicon material or other material which is the nonabsorptive material may be applied by the user to the tampon before insertion into the vagina area. As such, the silicon material can be a sleeve which is applied to the absorptive material. The system can be fabricated with the elastic expandable flexible silicon material suitably embedded or formed on the outside surface of the absorptive material in a manner that is securely anchored so that its inadvertent release or separation from the tampon is not possible.

The system improves comfort, minimizes infection and can avoid toxic shock syndrome.

Many modifications and variations are possible in the light of the above teaching. The foregoing is a description of the preferred embodiments of the invention and has been presented for the purpose of illustration and description. It is not intended to be exhaustive and so limit the invention to the precise form disclosed.

The internal area 13 can be a balloon like element which may be at least partly filled with one or more of a liquid and/or a resin and/or a sponge material. The area can be in a normally relatively dry state and the material may be vacuum packed in the central area. In use, the vacuum package is opened so that it becomes open to the environment and thereby expandable. Such expansion is promoted when the area 13 becomes moist so it can extend or expand.

The invention is to be determined by the following claims

What is claimed is:

1. A tampon comprising an elongated absorptive element having a central axis for location in a vagina, the elongated element having at least one elongated area which is expandable on the insertion of a fluid into the expandable area and thereby urge the absorptive area generally outwardly from the central axis and into a relatively closer engagement with the wall of the vagina.

2. A tampon comprising an elongated absorptive element having a central axis for location in the vagina, the elongated element having an outside surface, and the outside surface including at least one and preferably a plurality of nonabsorptive elements arranged in spaced relationship about the outside surface of the absorptive material.

3. A tampon as claimed in claim 1 including a pump for urging fluid, preferably air, into the tampon material, preferably into the elongated central area thereby to cause the expansion of the tampon outwardly and to engage the walls of the vagina.

4. A tampon as claimed in claim 2 including a pump for urging fluid, preferably air, into the tampon material, preferably into the elongated central area thereby to cause the expansion of the tampon outwardly and to engage the walls of the vagina.
